(19) United States
(12) Patent Application Publication
(10) Pub. No.: US 2007/0264293 A1
(43) Pub. Date: Nov. 15, 2007

(54) Encapsulated Fluorescent Whitening Compositions and Their Use in Personal Care Applications

(76) Inventors: Stewart Todd Elder, Butler, NJ (US); Christina Ligia Andrianov, Hawthorne, NJ (US)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/662,036

(22) PCT Filed: Aug. 31, 2005
(86) PCT No.: PCT/EP05/54269

§ 371 Date: Mar. 6, 2007

Related U.S. Application Data
(60) Provisional application No. 60/608,650, filed on Sep. 10, 2004.

(51) Int. Cl. A61K 9/14; A61K 8/02; A61Q 1/06; A61Q 1/12; A61Q 17/04; A61Q 19/02; A61Q 9/02

(52) U.S. Cl. 424/401; 424/490; 424/59; 424/64; 424/69; 424/78.03

(57) ABSTRACT

Microcapsules of a fluorescent whitening agent in a transparent or translucent polymer formed from a mixture of hydrophobic and ionic monomers that are capable of forming a homopolymer of glass transition temperature in excess of 50° C., wherein the fluorescent whitening agent is not bonded to the surface of a particulate substrate, provide attractive aesthetic effects when incorporated into personal care or cosmetic compositions.

### FIELD OF THE INVENTION

This invention relates to compositions containing encapsulated fluorescent whitening agents (FWAs) and their use in personal care applications. More particularly it relates to microcapsules comprising at least one fluorescent whitening agent and their preparation, compositions comprising microcapsules containing at least one fluorescent whitening agent and use thereof in personal care applications.

### BACKGROUND OF THE INVENTION

Various methods for making capsules have been proposed in the literature. For instance it is known to encapsulate hydrophobic liquids by dispersing the hydrophobic liquid into an aqueous medium containing a melamine formaldehyde pre-condensate and then reducing the pH resulting in an impervious aminoplast resin shell wall surrounding the hydrophobic liquid. Variations of this type of process are described in GB-A-2073132, AU-A-27028/88 and GB-A-1507739, in which the capsules are preferably used to provide encapsulated inks for use in pressure sensitive carbonless copy paper.

However, although capsules based on melamine formaldehyde resins are both impervious and durable, they tend to suffer the disadvantage that they are less impermeable at elevated temperatures. In addition there is also a risk that at elevated temperatures formaldehyde can be evolved. This is undesirable, particularly in a cosmetic application.

Typical techniques for forming a polymer shell are described in, for instance, GB 1,275,712, 1,475,229 and 1,507,739, DE 3,545,803 and U.S. Pat. No. 3,591,090.

U.S. Pat. No. 5,382,433 and published PCT Application WO 98/5002 describe the use of a cosmetic stick that contains microencapsulated pigment particles. The encapsulated pigment in the '433 patent is made by coacervation polymerization. The PCT application expands on this patent by including a volatile solvent in the cosmetic composition. The volatile solvent is present to minimize the gritty feel of the microencapsulated material.

U.S. Pat. No. 5,234,711 concerns methods of encapsulating pigment particles useful in manufacturing of cosmetic products. It is an objective of this disclosure to employ an encapsulation process for increasing the wettability, dispersibility and heat resistance of the pigment particles. The encapsulation method involves redox or free radical vinyl polymerization in an aqueous medium. The cosmetic products are especially directed to eyeliner pens.

Published European Patent Application 225,799 describes a microencapsulated solid non-magnetic colorant material in a liquid, gel, wax or low temperature melting solid carrier phase, which is encapsulated within a polymeric shell. Absorbed onto the shell is a silane or titanate coupling agent, which increases the oleophilicity of the surface of the solid colorant material.

Published European Patent Application 445,342 relates to a cosmetic composition comprising a pigment that has been formed by incorporating a solvate dye into a resin and admixing with a cosmetic carrier. The amount of pigment present is sufficient to provide an attractive cosmetic effect when applied to skin, nails or hair. Any cosmetically acceptable soluble dye can be used. Any resin may be used provided it can be pulverized to a fine powder. The solvate dye may be incorporated into the resin by adding it to the elasticized or molten resin, or by dissolving the dye in a solution of unpolymerized resin and a mutual solvent for the dye and the resin, then polymerizing the resin, or by contacting the dye with the resin. The dye-impregnated resin powders are said to be usable in a variety of cosmetic compositions.

WO 02/090445 addresses the problem of color retention and provides polymeric particles comprising a matrix polymer and colorant distributed throughout it. The matrix polymer is formed from a blend of monomers comprising a first monomer, which is an ethylenically unsaturated ionic monomer, which is a salt of a volatile counterion, and a second monomer, which is an ethylenically unsaturated hydrophobic monomer, which is capable of forming a homopolymer of glass transition temperature in excess of 50° C. Typical matrix polymers include copolymers that have been formed from styrene with ammonium acrylate. The polymeric particles exhibit good retention properties and are able to retain the colorant under a variety of conditions.

U.S. Patent Application Publication No. 2002/0192260 A1 discloses optically-activated particles for use in cosmetic preparations to reduce the visual perception of skin imperfections. The optically-activated particles are of various substrates such as nylons, acrylics, polyesters, other plastic polymers, natural materials, regenerated cellulose, metals and minerals, and having an optical brightener (fluorescent whitening agent) chemically bonded to the surface of the substrate particles to form integral units in the form of optically-activated particles for diffusing and emitting light to reduce the visual perception of cellulite, shadows, skin discolorations and wrinkles. Each of the optically-activated particles may be additionally encapsulated with a UV transparent coating, for example a polyoxymethylene urea, to increase the diffusion of light to further reduce the visual perception of cellulite, shadows, skin discolorations and wrinkles. According to page 3, paragraph [0029] of the disclosure, the optical brightener compound is chemically bonded to the substrate (e.g. a nylon spheroid particle) by covalent or ionic bonding, such that the optical brightener is inseparable from the nylon particle and becomes part of the finished optically-activated particle. In the present invention the optical brightener (FWA) is not bonded to surface of a particle.

Copending U.S. patent application Ser. No. 10/785,208 describes the use of a blend of microencapsulated colorants prepared as described in WO 02/090445 in cosmetic compositions. The blend produces a textured natural tone coloring when applied, or creates similar effects on or in the cosmetic product itself.

The aforementioned prior art does not describe the use of microencapsulated fluorescent whitening agents (FWAs) that are not bonded to surface of a particulate substrate in personal care applications.

Copending U.S. patent application Ser. No. 10/903,642 describes certain shatter-resistant microcapsules comprising at least one fluorescent whitening agent and their preparation, compositions comprising shatter-resistant microcapsules containing at least one fluorescent whitening agent and use thereof in personal care applications. However, the microcapsules therein are structurally different from those employed according to the present invention.

An object of the present invention is to provide a microcapsule comprising at least one fluorescent whitening agent wherein the fluorescent whitening agent is not bonded to the surface of a particulate substrate. Still another object is to provide a cosmetic composition comprising microcapsules containing said fluorescent whitening agent, whereby the compositions retain the FWA over extended periods, even when subjected to different environments. This is especially important when the FWAs are oil-soluble and particularly so when they are water-soluble, where it is generally difficult to permanently retain them. In a cosmetic composition, if the FWA is not permanently retained, this can impair the visual effect of the cosmetic after prolonged use.

### SUMMARY OF THE INVENTION

The present invention provides a microencapsulated fluorescent whitening agent wherein the fluorescent whitening agent is not bonded to the surface of a particulate substrate.

The present invention also provides a personal care or cosmetic composition that comprises a microencapsulated fluorescent whitening agent wherein the fluorescent whitening agent is not bonded to the surface of a particulate substrate.

The present invention also provides a method of masking or reducing the appearance of skin imperfections, which comprises application of a personal care or cosmetic formulation having an effective amount of a microencapsulated fluorescent whitening agent therein, wherein the fluorescent whitening agent is not bonded to the surface of a particulate substrate, to at least a part of the human body, in particular to the surface of the skin.

The microencapsulated FWAs according to the first aspect of the invention and use of the compositions process according to the second aspect of the invention enhance the visual performance of a personal care or cosmetic formulation. Furthermore the polymer matrix does not allow the entrapped FWA to be released even under prolonged use.

### DETAILED DESCRIPTION OF THE INVENTION

It has been found that transparent or translucent polymers formed from a special combination of hydrophobic and ionic monomers that are capable of forming a homopolymer of glass transition temperature in excess of 50° C., preferably greater than 60° C., more preferably greater than 80° C., exhibit considerably improved performance in regard to their impermeability to an entrapped fluorescent whitening agent. By hydrophobic monomer is meant that the monomer has a solubility in water of less than 5 g per 100 ml of water.

Thus, the present invention provides microcapsules of a fluorescent whitening agent in a transparent or translucent polymer formed from a mixture of hydrophobic and ionic monomers that are capable of forming a homopolymer of glass transition temperature in excess of 50° C., preferably greater than 60° C., more preferably greater than 80° C. wherein the fluorescent whitening agent is not bonded to the surface of a particulate substrate.

The glass transition temperature (T_{g}) for a polymer is defined in the Encyclopedia of Chemical Technology, Volume 19, fourth edition, page 891 as the temperature below which (1) the transitional motion of entire molecules and (2) the coiling and uncoiling of 40 to 50 carbon atom segments of chains are both frozen. Thus below its T_{g} a polymer would not exhibit flow or rubber elasticity.

The T_{g} of a polymer may be determined using Differential Scanning Calorimetry (DSC) by methods well known in the art.

Specific examples of hydrophobic monomers include styrene, methyl methacrylate, tertiary butyl methacrylate, phenyl methacrylate, cyclohexyl methacrylate and isobornyl methacrylate.

Without being limited to theory it is believed that the particular combination of ionic monomer and said hydrophobic monomer provides polymers with the right degree of hydrophilicity and hardness that seems to be responsible for the improvements in impermeability to the FWA.

It has been found that it is not possible to replace the hydrophobic monomers with ethylenically unsaturated carboxylic acid esters that are not capable of forming a homopolymer that has a glass transition temperature (T_{g}) of at least 50° C. without adversely increasing the permeability of the polymer. Preferably still the T_{g} should be at least 60° C. or even at least 80° C. For instance substituting the hydrophobic monomer of the present invention by other (meth) acrylic esters, for instance 2-ethylhexyl acrylate would be unsuitable. Best results are generally obtained by use of monomers that are capable of forming polymers of very high T_{g}, e.g., a T_{g} of at least 80° C.

Therefore less preferred products would be produced using ethyl acrylate or propyl acrylate as the hydrophobic monomer.

The ionic monomer may contain either anionic or cationic groups or alternatively may be potentially ionic, for instance in the form of an acid anhydride. Preferably the ionic monomer is an ethylenically unsaturated anionic or potentially anionic monomer. Suitable anionic monomers include (meth) acrylic acid, maleic acid, maleic anhydride, itaconic acid, itaconic acid anhydride, crotonic acid, (meth) allyl sulfonic acid, vinyl sulfonic acid and 2-acrylamido-2-methyl propane sulfonic acid. Preferred anionic monomers are carboxylic acids or acid anhydrides.

When the ionic monomer is anionic, for instance a carboxylic acid or anhydride, the volatile counterion may be ammonia or a volatile amine component. Thus the polymer may be produced in free acid form and then neutralized with an aqueous solution of ammonium hydroxide or a volatile amine, for instance ethanolamine.

Alternatively the polymer may be prepared by copolymerizing the ammonium or volatile amine salt of an anionic monomer with the hydrophobic monomer.

Generally the polymer may be prepared by any suitable polymerization process. For instance the polymer can be conveniently prepared by aqueous emulsion polymerization as described in EP-A-697423 or U.S. Pat. No. 5,070,136. The polymer can then be neutralized by the addition of an aqueous solution of ammonium hydroxide or a volatile amine.

In a typical polymerization process a blend of hydrophobic monomer and anionic monomer is emulsified into an aqueous phase that contains a suitable amount of emulsifying agent. Typically the emulsifying agent may be any commercially available emulsifying agent suitable for forming an aqueous emulsion.

Desirably these emulsifying agents will tend to be more soluble in the aqueous phase than in the water-immiscible monomer phase and thus will tend to exhibit a high hydrophilic lipophilic balance (HLB). Emulsification of the monomer may be effected by known emulsification techniques, including subjecting the monomer/aqueous phase to vigorous stirring or shearing or alternatively passing the monomer/aqueous phase through a screen or mesh. Polymerization may then be effected by use of suitable initiator systems, for instance a UV initiator or thermal initiator. A suitable technique of initiating the polymerization would be to elevate the temperature of the aqueous monomer emulsion to above 70 or 80° C. and then add between 50 and 1000 ppm ammonium of persulfate by weight of monomer.

Generally the copolymer has a molecular weight of up to 200,000 (Determined by GPC using standard industrial parameters). Preferably the polymer has a molecular weight of below 50,000, for instance 2,000 to 20,000.

Typically the monomer blend may contain at least 50% by weight of hydrophobic monomer, the remainder being made up of anionic monomer. Generally though the hydrophobic monomer will be present in amounts of at least 60% by weight.

Preferably the polymer is formed from a mixture of between 65 and 90% by weight of hydrophobic monomers, for instance around 70 or 75%, and between 10 and 35% by weight of anionic monomers, for instance around 25 or 30%.

A particularly preferred anionic polymer is a copolymer of styrene with ammonium acrylate, especially a polymer is formed from a mixture of between 65 and 90% by weight of styrene, and between 10 and 35% by weight of ammonium acrylate.

In an alternative version of the process of the present invention the ionic monomer may be cationic or potentially cationic, for instance an ethylenically unsaturated amine. In this form of the invention the volatile counterionic component is a volatile acid component. Thus in this form of the invention the polymer can be formed in an analogous way to the aforementioned anionic polymer, except that the anionic monomer is replaced by a cationic or potentially cationic monomer. Generally where the polymer is prepared in the form of a copolymer of a free amine and a hydrophobic monomer, it is neutralized by including a suitable volatile acid, for instance acetic acid, formic acid or even carbonic acid. Preferably the polymer is neutralized by a volatile carboxylic acid. The amount of cationic or potentially cationic monomer to hydrophobic monomer is generally the same as for the aforementioned anionic monomer.

The fluorescent whitening agent may be added before, during or after the copolymerization of the monomers.

The polymeric products can be further enhanced if the polymeric matrix is cross-linked. This cross-linking can be as a result of including a cross-linking step in the microencapsulation process. This can be achieved by including self cross-linking groups in the polymer, for instance monomer repeating units carrying a methylol functionality.

Preferably the cross-linking is achieved by including a cross-linking agent with the aqueous phase polymer. The cross-linking agents are generally compounds which react with functional groups on the polymer chain. For instance when the polymer chain contains anionic groups, suitable cross-linking agent may include aziridines, diepoxides, carbodiamides, silanes and multivalent metals, for instance aluminum, zinc or zirconium. Particularly preferred cross-linking agents are zinc carbonate and ammonium zirconium carbonate. Another particularly preferred class of cross-linking agents includes compounds which form covalent bonds between polymer chains, for instance silanes or diepoxides.

The cross-linking process desirably occurs during and after the dehydration step. Thus, where a cross-linking agent is included, it will generally remain dormant until the dehydration is started.

In one embodiment the fluorescent whitening agent is added after the copolymerization of the monomers but before the dehydration step. Thus the particles formed will entrap the fluorescent whitening agent.

Fluorescent whitening agents are substances that absorb light in the invisible ultraviolet region of the spectrum and reemit it in the visible portion of the spectrum, particularly in the blue to blue-violet wavelengths. This provides added brightness and can offset the darkening in areas of a substrate such as skin due to crevices or wrinkles.

The choice of the fluorescent whitening agent used in the present invention is not critical. The fluorescent whitening agent may be any FWA, for instance a water-soluble or an oil-soluble FWA.

The choice of the fluorescent whitening agent used in the present invention is not critical. It can be oil or water soluble and may be selected from a wide range of chemical classes such as 4,4′-bis-(triazinylamino)-stilbene-2,2′-disulfonic acids, 4,4′-bis-(triazol-2-yl)stilbene-2,2′-disulfonic acids, 4,4′-dibenzofuranyl-biphenyls, 4,4′-(diphenyl)-stilbenes, 4,4′-distyryl-biphenyls, 4-phenyl-4′-benzoxazolyl-stilbenes, stilbenzyl-naphthotriazoles, 4-styryl-stilbenes, bis-(benzoxazol-2-yl) derivatives, bis-(benzimidazol-2-yl) derivatives, coumarins, pyrazolines, naphthalimides, triazinyl-pyrenes, 2-styryl-benzoxazole or -naphthoxazoles, benzimidazole-benzofurans and oxanilides. Mixtures thereof of fluorescent whitening agents of the same or different chemical classes may be employed.

Microcapsules wherein the fluorescent whitening agent is selected from the group consisting of 4,4′-bis-(triazinylamino)-stilbene-2,2′-disulfonic acids, 4,4′-dibenzofuranyl-biphenyls and mixtures thereof are especially preferred.

It is noted that fluorescent whitening agents may exhibit a green or bluish cast in concentrated form. This can be counteracted by the use of appropriate levels of mixtures of fluorescent whitening agents, particularly mixtures which contain fluorescent whitening agents having a more reddish cast. Therefore mixtures of fluorescent whitening agents of the same or different chemical classes may be advantageously employed.

The process to prepare the microcapsules of the present invention comprises dispersing an aqueous solution of the polymer described above and a fluorescent whitening agent into a water-immiscible liquid. Typically the water-immiscible liquid is an organic liquid or blend of organic liquids. A preferred organic liquid is a mixture of a non-volatile paraffin oil and a volatile paraffin oil. The two oils may be used in equal proportions by weight, but generally it is often preferred to use the non-volatile oil in excess, for instance greater than 50 to 75 parts by weight of the non-volatile oil to 25 to less than 50 parts by weight of the volatile oil.

In this it is desirable to include a polymeric amphipathic stabilizer in the water-immiscible liquid. The amphipathic stabilizer may be any suitable commercially available amphipathic stabilizer, for instance HYPERMER® (available from ICI). Suitable stabilizers also include the stabilizers described in WO 97/24179.

Although it is possible to include other stabilizing materials in addition to the amphipathic stabilizer, such as surfactants, it is generally preferred that the sole stabilizing material is the amphipathic stabilizer.

In the process of the present invention the dehydration step can be achieved by any convenient means. Desirable dehydration can be effected by subjecting the dispersion in oil to vacuum distillation. Generally this will require elevated temperatures, for instance temperatures of 30° C. or higher. Although it may be possible to use much higher temperatures, e.g. 80 to 90° C., it is generally preferred to use temperatures of below 60 or 70° C. Subsequent heating without vacuum above 80° C., e.g. 90-120° C., may be desirable to complete the crosslinking process.

Instead of vacuum distillation, it may be desirable to effect dehydration by spray drying. Suitably this can be achieved by the spray drying process described in WO 97/34945.

The dehydration step removes water from the aqueous solution of polymer and also the volatile counterion component, resulting in a slurry of microcapsules containing therein the FWA which is distributed within the microparticles in the dry water-immiscible liquid. Said microcapsules can be collected and dried. They are insoluble and non-swellable in water.

Generally the average particle size diameter of the particles is less than about 200 microns. Usually the average particle size diameter tends to be smaller, for instance less than 150 or 100 microns, and typically the average particle diameter will be between 750 nanometers and 50 microns.

Preferably the average particle size diameter is in the range 1 to 150 microns, especially between 10 and 30 microns.

Average particle size is determined by a Coulter particle size analyzer according to standard procedures well documented in the literature.

It has now been found that applying a personal care or cosmetic comprising at least one microencapsulated FWA incorporated therein produces desirable effects upon application to the human body. The microparticles according to the invention are able to both scatter and reemit white light in a diffuse manner in order to reduce the visual appearance and perception of skin imperfections, such as shadows, skin discolorations, wrinkles and cellulite when applied to at least a part of the body, for example to the surface of the skin. Hence the present invention additionally relates to a method of masking or reducing the appearance of skin imperfections, which comprises applying a solid or liquid personal care or cosmetic formulation having an effective amount of at least one microencapsulated FWA as described above incorporated therein to the surface of the skin.

The personal care composition according to the invention comprises from 0.0001 to 10% by weight, for example from 0.001 to 8% by weight, and especially from 0.005 to 5% by weight based on the total weight of the composition, of the microencapsulated FWA as well as a cosmetically tolerable carrier or adjuvant which is other than, or in addition to water. While water is cosmetically tolerable, and in most instances will also be present, the phrase “a cosmetically tolerable carrier or adjuvant” is intended to refer to at least one substance other than water that is customarily employed in personal care or cosmetic compositions.

Depending on the intended use, the preferred average diameters will vary. For example one embodiment of this invention may be a liquid facial cosmetic formulation comprising the microencapsulated FWA described above and having a preferred particle size range of between 10 and 30 microns. Another embodiment may be a lipstick formulation comprising the microencapsulated FWA described above having preferred particle sizes of between 1 and 10 microns.

The personal care or cosmetic preparation according to the invention may be formulated as a water-in-oil or oil-in-water emulsion, as an alcoholic or alcohol-containing formulation, as a vesicular dispersion of an ionic or non-ionic amphiphilic lipid, as a gel, or a solid stick. Preferably the cosmetic preparation is in the form of a liquid.

As a water-in-oil or oil-in-water emulsion, the cosmetically tolerable adjuvant contains preferably from 5 to 50% of an oily phase, from 5 to 20% of an emulsifier and from 30 to 90% of water. The oily phase may contain any oil suitable for cosmetic formulations, e.g. one or more hydrocarbon oils, a wax, a natural oil, a silicone oil, a fatty acid ester or a fatty alcohol. Examples are mineral oil, castor oil, cyclomethicone, dimethicone, dimethicone copolyol, phenyl trimethicone, trimethyl pentaphenyl trisiloxane, caprylic/capric triglyceride, isostearyl stearoyl stearate, octyldodecyl erucate, triisostearyl citrate, triisostearyl trilinoleate, pentaerythrityl tetraisononanoate, isopropyl myristate, isopropyl palmitate, octyl palmitate, diisostearyl malate, diethyl sebacate and diisopropyl adipate.

Cosmetic liquids may contain mono- or polyols such as ethanol, isopropanol, propylene glycol, hexylene glycol, glycerol or sorbitol.

Cosmetic formulations according to the invention may be contained in a wide variety of cosmetic preparations. Especially the following preparations, for example, come into consideration:
shaving preparations, e.g. aftershave lotions or aftershave creams;
skin-care preparations, e.g. skin emulsions, multi-emulsions or skin oils and body powders;
cosmetic personal care preparations, e.g. facial make-up in the form of lipsticks, day creams, facial lotions, and creams; and
light-protective preparations, such as sun tan lotions, creams and oils, sun blocks and pretanning preparations.

Depending upon the form of the personal care preparation, it will comprise, in addition to the encapsulated FWAs, further constituents, for example sequestering agents, non-encapsulated colorants or encapsulated colorants, perfumes, thickening or solidifying (consistency regulator) agents, emollients, non-encapsulated UV absorbers, skin-protective agents, antioxidants and preservatives.

The term “perfume” or “fragrance” as used herein refers to odoriferous materials which are able to provide a pleasing fragrance to fabrics, and encompasses conventional materials commonly used in cosmetic compositions to counteract a malodor in such compositions and/or provide a pleasing fragrance thereto. The perfumes are preferably in the liquid state at ambient temperature, although solid perfumes are also useful, particularly cyclodextrin/-perfume inclusion complexes for controlled release. Included among the perfumes contemplated for use herein are materials such as aldehydes, ketones, esters and the like which are conventionally employed to impart a pleasing fragrance to liquid and solid personal care or cosmetic compositions. Naturally occurring plant and animal oils are also commonly used as components of perfumes. Accordingly, the perfumes useful for the present invention may have relatively simple compositions or may comprise complex mixtures of natural and synthetic chemical components, all of which are intended to provide a pleasant odor or fragrance when applied to fabrics. The perfumes used in personal care or cosmetic compositions are generally selected to meet the normal requirements of odor, stability, price and commercial availability. The term “fragrance” is often used herein to signify a perfume itself, rather than the aroma imparted by such perfume.

As a further customary additive, the personal care or cosmetic compositions may also comprise at least one sequestering agent. Sequestering agents act to sequester (chelate) metal ions. Said sequestering agents may be present at a level of up to 0.5%, more preferably from 0.005% to 0.25%, most preferably from 0.01% to 0.1 wt-%, based on the total weight of the personal care or cosmetic composition.

Chelating components are present at a level of up to 0.5%, more preferably from 0.005% to 0.25%, most preferably from 0.01% to 0.1 wt-%, based on the total weight of the composition.

Suitable chelating components for use in the present invention are selected from the group consisting of amino carboxylic acids, organo aminophosphonic acid compounds, and mixtures thereof.

Chelating components, which are acidic in nature, having for example phosphonic acid or carboxylic acid functionalities, may be present either in their acid form or as a complex/salt with a suitable counter cation such as an alkali or alkaline metal ion, ammonium, or substituted ammonium ion, or any mixtures thereof. Preferably any salts/complexes are water-soluble. The molar ratio of said counter cation to the chelating component is preferably at least 1:1.

Suitable chelating components for use herein include the amino carboxylic acids such as ethylenediamine-N,N′-disuccinic acid (EDDS), ethylenediamine tetraacetic acid (EDTA), N-hydroxyethylenediamine triacetic acid, nitrilotriacetic acid (NTA), ethylene diamine tetrapropionic acid, ethylenediamine-N,N′-diglutamic acid, 2-hydroxypropylenediamine-N,N′-disuccinic acid, triethylenetetraamine hexacetic acid, diethylenetriamine pentaacetic acid (DETPA), trans 1,2diaminocyclohexane-N,N,N′,N′-tetraacetic acid or ethanoldiglycine.

Other suitable chelating components for use herein include the organo aminophosphonic acids such as ethylenediamine tetrakis (methylenephosphonic acid), diethylene triamine-N,N,N′,N″,N″-pentakis (methylene phosphonic acid) (DETMP), 1-hydroxyethane 1,1-diphosphonic acid (HEDP) or hydroxyethane dimethylenephosphonic acid.

Mixtures of chelating components can also be used.

Especially preferred is ethylenediamine-N,N′-disuccinic acid (EDDS), most preferably present in the form of its S,S isomer, which is preferred for its biodegradability profile.

The cosmetic or personal care formulation according to the present invention may comprise 0-50 wt-% of at least one alcohol.

Suitable alcohols include dihydric alcohols especially those compounds having from 2 to 6 carbon atoms in the alkylene moiety, such as ethylene glycol, 1,2- or 1,3-propanediol, 1,3-, 1,4- or 2,3-butanediol, 1,5-pentanediol or 1,6-hexanediol.

Preference is given to 1,2-propanediol (propylene glycol).

Preferred monohydric alcohols are ethanol, n-propanol and isopropanol and mixtures of these alcohols.

Compositions according to the invention may be prepared by physically blending the microcapsules as described above containing one or more fluorescent whitening agents into personal care formulations by methods which are well known in the art. The examples illustrate several such methods.

The present invention additionally relates to a method of masking or reducing the appearance of skin imperfections, which comprises applying a solid or liquid personal care or cosmetic formulation having an effective amount of the microencapsulated FWA according to the invention to the surface of the skin.

In one embodiment of the method, the personal care or cosmetic formulation comprises from 0.0001 to 10% by weight, for example from 0.001 to 8% by weight, and especially from 0.005 to 5% by weight based on the total weight of the formulation, of the microencapsulated FWAs.

In one embodiment of the method, the personal care or cosmetic composition comprises a blend of microcapsules as described above containing different microencapsulated fluorescent whitening agents that are individually provided in separate polymers. In another, the personal care or cosmetic composition comprises microcapsules as described above containing a blend of at least two different fluorescent whitening agents that are embedded in a single polymer.

In one embodiment of the method, the personal care or cosmetic composition is formulated as a water-in-oil or oil-in-water emulsion, as an alcoholic or alcohol-containing formulation, as a vesicular dispersion of an ionic or non-ionic amphiphilic lipid, as a gel, or a solid stick.

In various embodiments of the method, the personal care or cosmetic composition is in the form of a shaving preparation, a skin-care preparation, a cosmetic personal care preparation, or a light-protective preparation.

The following examples describe certain embodiments of this invention, but the invention is not limited thereto. It should be understood that numerous changes to the disclosed embodiments could be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated.

### EXAMPLE 1

An aqueous phase is prepared by milling 10 g Tinopal AMS-GX® (available from Ciba Specialty Chemicals) powder into 225 g of a 25% solution of a polymer of styrene-acrylic acid ammonium salt (65/35 weight % monomers ratio, molecular weight about 6,000). To the resulting smooth aqueous dispersion is added 8.6 g of a 50% aqueous solution of ammonium zirconium carbonate.

Separately, an oil phase is prepared by diluting 22 g of 20% Lopol® polymeric emulsion stabilizer (available from Ciba Specialty Chemicals) with 675 g of Isopar Go solvent (available from Exxon Mobil). This oil mixture is charged into a 1-litre flask equipped with a mechanical agitator and vacuum distillation capabilities.

The aqueous phase prepared above is added to the mechanically agitated oil phase to form a water-in-oil suspension with droplets having average diameters of approx. 60 microns. The flask contents are warmed to 60° C. while the water/Isopar G mixture is distilled under reduced pressure. The distillation is continued until no further water is collected in the distillate. Then the temperature of the flask allowed to rise to 95° C. without vacuum. The slurry of formed polymeric beads in Isopar G is held at 95° C. for 1 hour to drive off the ammonia and to crosslink the carboxylated styrene-based copolymer.

The flask contents are cooled to room temperature and then the FWA slurry is filtered through No. 1 filter paper under vacuum. The resultant FWA beads are then dried at 110° C. for 2 hours. The final product is dried polymeric spherical beads having average particle diameters of 60 microns containing approximately 15% FWA.

### FORMULATION EXAMPLE 1

### Oil-in-Water Cream

| Phase | Ingredient | Amount [wt-%] | Amount [wt-%] |
|---|---|---|---|
| A | Deionized Water | 76.35 | 80.35 |
| | Hydroxyethylcellulose | 1.50 | 1.50 |
| | Methylparaben, USP | 0.30 | 0.30 |
| B | C₁₂-C₁₅ Alkyl Octanoate | 3.00 | 3.00 |
| | Trioctyldodecyl Citrate | 2.00 | 2.00 |
| | Ethylhexyl Palmitate | 3.00 | 3.00 |
| | Glyceryl Stearate | 1.00 | 1.00 |
| | Stearic Acid | 2.00 | 2.00 |
| | Sorbitan Oleate | 0.80 | 0.80 |
| | Polysorbate 80 | 0.15 | 0.15 |
| | Propylparaben, USP | 0.10 | 0.10 |
| C | Deionized Water | 2.00 | 2.00 |
| | Triethanolamine | 1.50 | 1.50 |
| D | Encapsulated FWA of | 5.00 | 1.00 |
| | Example 1 | | |
| E | Deionized Water | 1.00 | 1.00 |
| | Diazolidinyl Urea | 0.30 | 0.30 |

Procedure

In a suitable vessel the water and the hydroxyethylcellulose of phase A are mixed using a homogenizer for 30 minutes and heated to 75-80° C. Then the methyl paraben of phase A is added and mixed for about 5 minutes, maintaining the same temperature as above. The ingredients of phase B are premelted at 75-80° C. and mixed in a separate vessel. When a uniform liquid solution is obtained phase B is added to phase A using a high-speed homogenizer. The emulsion is homogenized for 15-20 minutes at 75-80° C. Phase C is then added to the vessel using the homogenizer. The heating process is stopped and the mixture is allowed to gradually cool down. At 55-60° C. phase D is added using a Lightning high speed mixer. At 45° C. phase E is added to the vessel. Mixing is stopped when room temperature is achieved.

This results an O/W cream with good overall properties.

### FORMULATION EXAMPLE 2

### Liquid Makeup—Oil-in-Water Foundation

| Phase | Ingredient | Amount [wt-%] |
|---|---|---|
| A | Deionized Water | 53.88 |
| | Sodium Hydroxide 10% solution | 1.30 |
| | Dimethicone Copolyol | 0.10 |
| B | 80% Titanium Dioxide/Talc | 7.50 |
| | 80% Yellow Iron Oxide/Talc | 2.25 |
| | 80% Red Iron Oxide/Talc | 1.38 |
| | 80% Black Iron Oxide/Talc | 0.25 |
| | Talc | 0.72 |
| C | Butylene Glycol | 4.00 |
| | Magnesium Aluminum Silicate | 1.00 |
| D | Butylene Glycol | 2.00 |
| | Cellulose Gum | 0.10 |
| E | Methylparaben | 0.10 |
| F | Di-PPG-3 Myristyl Ether Adipate | 14.00 |
| | Dioctyl Maleate | 4.00 |
| | Steareth-10 | 2.00 |
| | Steareth-2 | 0.50 |
| | Cetyl Alcohol | 0.62 |
| | Dicetyl Phosphate, Ceteth-20 Phosphate, | 4.00 |
| | Cetearyl Alcohol | |
| | Propylparaben | 0.10 |
| G | Encapsulated FWA of Example 1 | 0.02 |
| H | DMDM Hydantoin | 0.18 |
| | Total | 100.00 |

In suitable vessel phase A is heated to 75-80° C. and mixed well using a homogenizer. Pre-ground phase B is added to A and homogenized for 1 hour. Pre-mixed phase C is added. Pre-mixed phase D is added and the mixture is homogenized for 30 minutes maintaining the temperature at 75-80° C. Phase E is added under the same conditions.

In a separate vessel phase F is melted until clear and uniform. Phase F is added using a homogenizer for 15 minutes. The mixture is cooled to 40-45° C. Phase G and H are added. Then the mixture is cooled to room temperature.

This results a liquid O/W makeup foundation with good overall properties.

### FORMULATION EXAMPLE 3

### Hard Surface Cleaner Formulation

| Ingredient | Amount [wt-%] |
|---|---|
| Octyl Alcohol Ethoxylate, 4 EO, 100% | 4.00 |
| Alkylpolyglycoside C₈-C₁₀, 62% | 2.00 |
| Isopropyl Alcohol | 3.00 |
| Rheovis ATA ® (from Ciba Specialty Chemicals) | 1.70 |
| Encapsulated FWA of Example 1 | 0.02 |
| Potassium Hydroxyde, 50% | to pH 8 |
| Deionized Water | to 100% |

The first three items are added to the water and stirred until uniform solution is obtained. The Rheovis ATA is slowly added to the main batch. The pH is adjusted to 8 using potassium hydroxide. The encapsulated FWA is added to the main batch and stirred well.

This results a hard surface cleaner formulation with good overall properties.

### FORMULATION EXAMPLE 4

### Household Fabric Softener

| Phase | Ingredient | Amount [wt-%] |
|---|---|---|
| A | Deionized Water | 94.68 |
| B | Dihydrogenated tallow dimethylammonium | 4.00 |
| | CI (75%) | |
| C | Nonionic thickener/stabilizer (Acusol 880 ® | 0.50 |
| | from Rohm and Haas Co.) | |
| D | Colorant | 0.30 |
| | Fragrance | 0.50 |
| | Encapsulated FWA of Example 1 | 0.02 |

Phase A is heated to 65-70° C. in a suitable vessel. Phase B is heated to 70° C. in a separate vessel until uniform; then added to A using high shear mixing. Phase C is added to the AB mixture, which is then cooled to 45° C. The ingredients of phase D are added one by one to the stirred mixture. The mixture is then cooled to room temperature.

This results a household fabric softener with good overall properties.

What is claimed is:

1. Microcapsules of a fluorescent whitening agent in a transparent or translucent polymer formed from a mixture of hydrophobic and ionic monomers that are capable of forming a homopolymer of glass transition temperature in excess of 50° C. wherein the fluorescent whitening agent is not bonded to the surface of a particulate substrate.

2. Microcapsules according to claim 1, wherein the polymer is formed from a mixture of hydrophobic and ionic monomers that are capable of forming a homopolymer of glass transition temperature in excess of 80° C.

3. Microcapsules according to claim 1, wherein the polymer is formed from a mixture of hydrophobic and anionic monomers.

4. Microcapsules according to claim 1, wherein the polymer is formed from a mixture of between 65 and 90% by weight of hydrophobic monomers and between 10 and 35% by weight of anionic monomers.

5. Microcapsules according to claim 1, wherein the polymer is cross-linked.

6. Microcapsules according to claim 1, wherein the fluorescent whitening agent is selected from the group consisting of 4,4′-bis-(triazinylamino)-stilbene-2,2′-disulfonic acids, 4,4′-bis-(triazol-2-yl)stilbene-2,2′-disulfonic acids, 4,4′-dibenzofuranyl-biphenyls, 4,4′-(diphenyl)-stilbenes, 4,4′-distyryl-biphenyls, 4-phenyl-4′-benzoxazolyl-stilbenes, stilbenzyl-naphtho-triazoles, 4-styryl-stilbenes, bis-(benzoxazol-2-yl) derivatives, bis-(benzimidazol-2-yl) derivatives, coumarins, pyrazolines, naphthalimides, triazinyl-pyrenes, 2-styryl-benzoxazole or -naphthoxazoles, benzimidazole-benzofurans and oxanilides, and mixtures thereof of fluorescent whitening agents of the same or different chemical classes.

7. Microcapsules according to claim 1, wherein the fluorescent whitening agent is selected from the group consisting of 4,4′-bis-(triazinylamino)-stilbene-2,2′-disulfonic acids, 4,4′-dibenzofuranyl-biphenyls and mixtures thereof.

8. Microcapsules according to claim 1, wherein the average particle size diameter is in the range 1 to 150 microns.

9. A process to prepare microcapsules according to claim 1, which comprises dispersing an aqueous solution of a transparent or translucent polymer formed from a mixture of hydrophobic and ionic monomers that are capable of forming a homopolymer of glass transition temperature in excess of 50° C. and a fluorescent whitening agent into a water-immiscible liquid, optionally in the presence of a polymeric amphipathic stabilizer, dehydrating the resulting dispersion and separating the microcapsules therein from the water-immiscible liquid.

10. A solid or liquid personal care or cosmetic composition, which comprises microcapsules as defined in claim 1 and a cosmetically tolerable carrier or adjuvant which is other than, or in addition to water.

11. A solid or liquid composition according to claim 10, which comprises from 0.0001 to 10% by weight of the microcapsules.

12. A composition according to claim 10, which is formulated as a water-in-oil or oil-in-water emulsion, as an alcoholic or alcohol-containing formulation, as a vesicular dispersion of an ionic or non-ionic amphiphilic lipid, as a gel, or a solid stick.

13. A composition according to claim 10, wherein the personal care or cosmetic composition is in the form of a shaving preparation, a skin-care preparation, a cosmetic personal care preparation or a light-protective preparation.

14. A composition according to claim 10, wherein the shaving preparation is an aftershave lotion or aftershave cream, the skin-care preparation is a skin emulsion, a multi-emulsion, a skin oil or a body powder, the cosmetic personal care preparation is a facial make-up in the form of lipstick, a day cream, a facial lotion or cream, and the light-protective preparation is a sun tan lotion, cream or oil, a sun block or a pretanning preparation.

15. A composition according to claim 10, which further comprises at least one further constituent selected from the group consisting of sequestering agents, non-encapsulated or encapsulated colorants, perfumes, thickening or solidifying (consistency regulator) agents, emollients, non-encapsulated or encapsulated UV absorbers, skin-protective agents, antioxidants and preservatives.

16. A method of masking or reducing the appearance of skin imperfections, which comprises applying a solid or liquid personal care or cosmetic formulation having an effective amount of microcapsules as defined in claim 1 to the surface of the skin.

17. A method according to claim 16, wherein the personal care or cosmetic composition comprises a blend of microcapsules containing different fluorescent whitening agents that are individually provided in separate polymers.

18. A method according to claim 16, wherein the personal care or cosmetic composition comprises microcapsules containing a blend of at least two different fluorescent whitening agents that are embedded in a single polymer.

19. A method according to claim 16, wherein the personal care or cosmetic composition is in the form of a skin-care preparation, a cosmetic personal care preparation or a light-protective preparation.
